(19) United States
(12) Patent Application Publication
(10) Pub. No.: US 2013/0084575 A1
(43) Pub. Date: Apr. 4, 2013

(54) METHOD AND DEVICE FOR REPLICATING A CELL COLONY IN CULTURE FOR EARLY ANALYSIS

(71) Applicant: The University of North Carolina at Chapel Hill, Chapel Hill, NC (US)

(72) Inventors: Nancy Allbritton, Chapel Hill, NC (US); Christopher Sims, Chapel Hill, NC (US); Wei Xu, Tianjin (CN)

(73) Assignee: The University of North Carolina at Chapel Hill, Chapel Hill, NC (US)

(21) Appl. No.: 13/629,955

(22) Filed: Sep. 28, 2012

Related U.S. Application Data
(60) Provisional application No. 61/542,948, filed on Oct. 4, 2011.

(51) Int. Cl. C12Q 1/04; G01N 27/62; G01N 33/53; C12M 1/00; C12Q 1/68

(52) U.S. Cl. 435/6.12; 435/305.1; 435/34; 435/6.1; 435/7.1

(57) ABSTRACT

The present invention provides an apparatus comprising, in combination: (a) a cell culture plate, the cell culture plate comprising a first substrate and a plurality of cell carriers on the substrate in a first pattern; and (b) a cell replication plate, the cell replication plate comprising a second substrate and a plurality of cell sampling posts on the second substrate in a second pattern corresponding to the first pattern. Each of the sampling posts is configured to align with a respective one of the cell carriers in a position in which cells growing on the cell carrier propagate onto the sampling post; so that a plurality of distinct cell colonies growing on the cell culture plate are replicated on the cell replication plate. Methods of using the same are also described.

### RELATED APPLICATIONS

This application claims the benefit of U.S. Provisional Patent Application Ser. No. 61/542,948, filed Oct. 4, 2011, the disclosure of which is incorporated by reference herein in its entirety.

### STATEMENT OF FEDERAL SUPPORT

This invention was made with Government support under grant nos. RO1HG004843 and RO1EB012549 from the National Institutes of Health. The US Government has certain rights to this invention.

### BACKGROUND OF THE INVENTION

The molecular engineering of specific genes is an important and common effort in academic and industrial laboratories. In most instances, modification of the genetic material is accomplished by transfecting cells with exogenous DNA. Although modern techniques can often achieve a high percentage of cells taking up and expressing the DNA for short time periods (transient transfection), it is a fairly rare event for the DNA to be incorporated into the host genome for stable reproduction and transfer to daughter cells. Indeed, the incidence of stably transfected cells may be as low as 1 in 10,000 or less. As a result, diligent efforts to select small numbers of cells from a large population are required.

To create or maintain a stably expressing cell line, transfected cells are typically subjected to antibiotic selection. Although antibiotic selection is toxic to cells failing to maintain the co-transfected resistance gene, some non-transfected cells can survive the process and some stable transfectants fail to survive with mixed populations being the result. Since the desire is to achieve a homogeneous population of cells carrying the desired phenotype, antibiotic selection combined with cloning is often performed using cloning rings or limiting dilution, two time- and manpower-intensive procedures. The process of antibiotic selection and cloning typically requires 4-6 weeks before adequate cell numbers can be grown for analysis. The rapid loss of expression seen in many transient transfection protocols suggests the possibility that stable clones might be generated more rapidly than traditional approaches if small clonal colonies could be isolated and expanded in the absence of a toxic selection pressure within days of transfection. This feat requires a means to efficiently detect transfection at early stages of colony formation.

Biochemical assays that enable molecular characterization of cells typically require destruction of the cells; thereby making it necessary to grow cells in sufficient numbers to split so that one fraction is subjected to the assay while the remainder is retained in culture for use after the target population is confirmed or identified. This is a particular concern with cloning efforts in which picking optimal colonies from a pool of randomly generated cell lines is required to create the desired cell line. Due to the need for compatibility with fastidious cell types and costs related to time and manpower, picking of colonies remains a costly, yet critical, effort in cell-line generation. To accomplish this activity requires tedious colony-forming assays using limiting dilution or cloning rings in conjunction with isolation and collection after trypsinization and pipet aspiration. Cells must be cultured for weeks to reach adequate numbers to allow these manipulations and colony splitting. Cross-contamination and limitations on the numbers of colonies that can be obtained are additional obstacles.

High-throughput efforts in the pharmaceutical industry have motivated the creation of a variety of automated platforms for clone selection. Robotic colony picking instruments are available (e.g. CellCelector™, Aviso, Jena, Germany, and ClonePix, Genetix, Hampshire, UK); however, these technologies are too costly for most users and suffer limitations beyond their price. The tools were initially developed for aspirating bacterial and yeast colonies from agar plates. Their use for mammalian cells is limited to cell types that grow as loosely adherent clusters in a semi-solid methylcellulose medium, and are not appropriate for most mammalian cell types. The viability of cells obtained in this manner is also limited, so that robotic clone picking is generally only used with bacterial or robust tumor cell lines

In recent years, microfabricated devices have been developed for culture and screening of cells; although, retrieval of viable cells from these devices has in general not been the primary aim. Techniques based on microfluidic, dielectrophoretic, optical- or magnetic-based devices have been extensively reported, although almost all have been directed at single cells, not colonies. A few reports have involved whole colony retrieval and a very limited number have described colony replication or isolation of partial colonies. Individual colonies have been grown in microwells or on patterned arrays, and then manually retrieved with a pipette.

We have previously described microarrays of pedestal-like elements termed pallets on which clonal colonies could be produced and the colonies individually collected with high viability. (see, e.g., Allbritton et al., U.S. Pat. No. 7,759,119; Bachman et al., U.S. Pat. No. 7,695,954; Wang et al., US Patent Application Pub. No. US2007/0128716; Allbritton et al., WO 2010/093766; Allbritton et al., WO 2011/103143). This work was extended by modification of the geometry to encourage expansion of colonies over a geographic area created by the surfaces of multiple pallets. By removing one of the pallets, clonal colonies composed of as few as 4 cells could be effectively split with cells from each colony obtained for biochemical assay and continued culture (Allbritton et al., WO2010/068743). However, the requirement for releasing individual pallets, particularly for a destructive assay, requires the addition of potentially complicated pallet release apparatus. And, disruption of the colony by removal of an individual pallet is potentially problematic for small colonies by killing the remaining cells or for large colonies by increased difficulty in releasing and collecting a single pallet containing the cell sample. In addition, the release of single elements from the array must typically done in a serial manner, complicating the collection process and increasing the time for release. Accordingly, there is a need for new ways to sample individual colonies grown in a culture.

### SUMMARY OF THE INVENTION

A first aspect of the invention is an apparatus comprising, in combination:

(a) a cell culture plate, the cell culture plate comprising a first substrate and a plurality of cell carriers on the substrate in a first pattern;

(b) a cell replication plate, the cell replication plate comprising a second substrate and a plurality of cell sampling posts on the second substrate in a second pattern corresponding to the first pattern, each of the sampling posts configured to align with a respective one of the cell carriers in a position in which cells growing on the cell carrier propagate onto the sampling post; so that a plurality of distinct cell colonies growing on the cell culture plate are replicated on the cell replication plate.

In some embodiments, the cell culture plate and the cell replication plate are positioned on one another, with the cell carriers and the sampling posts facing one another.

In some embodiments, the cell culture plate and the cell replication plate each have an indexing element formed thereon or connected thereto for aligning the cell carriers and the sampling posts.

In some embodiments, the cell carriers are releasably connected to the substrate.

In some embodiments, the first substrate is flexible.

In some embodiments, the cell carriers are rigid.

In some embodiments, the cell carriers are transparent.

In some embodiments, the cell carriers comprise microcups.

In some embodiments, the cell culture plate further comprises: trapped gas regions (e.g., hydrophobic cavities) on the substrate, with the plurality of cell carriers separated from one another by the trapped gas regions; and/or walls on the substrate, with the plurality of cell carriers separated from one another by the walls.

In some embodiments, the cell carriers have heights of at least 2 micrometers, up to 500 micrometers; and/or wherein the cell carriers have maximum widths of at least 5 micrometers, up to 1000 micrometers; the cell carriers are included on the first substrate at a density of from 0.1 or 0.2 to 10 or 20 carriers per square millimeter; the posts have heights of at least 10 micrometers, up to 1000 micrometers; and/or the posts have maximum widths at the tip thereof of at least 1 micrometer, up to 500 micrometers; and/or the posts are included on the second substrate at a density the same as the density of the cell carriers on the second substrate.

While the present invention is described as a combination above and below, it will be appreciated that the individual elements (e.g., the cell culture plate; the cell replication plate) are also an aspect of the invention individually or as a subcombination thereof.

Non-limiting examples of the present invention are set forth in greater detail in the drawings herein and the specification set forth below. The disclosures of all United States Patent references cited herein are to be incorporated by reference herein in their entirety.

### BRIEF DESCRIPTION OF THE DRAWINGS

FIG. 1. Schematic of the protocol for use of the pallet and printing arrays for identifying and cloning of desired cells. A) Cross sectional view of the pallet array plated with single cells (small black circles). The larger squares at the edge of the array represent the alignment structures (schematic is not to scale). B) The cells on the pallet array have expanded into clonal colonies. C) Cross sectional view of the printing array. The grooved polygons at the edges of the array represent the alignment structures. Shown also is the fluid reservoir on the backside of the array substrate used to weight the array after mating. D) Cross sectional view of the mated arrays. E) Cells are seen migrating along the posts to the printing array. F) The arrays are separated with the pallet array being returned to culture while the printing array is subjected to an assay for target identification. G) Target colony(s) are released and collected.

FIG. 2. Scanning electron micrographs of the arrays. A) The pallet array. The individual pallets are 150 μm (L)×150 μm (W)×120 μm (H) with a 150 μm inter-pallet gap. B) The printing array. The base pallet is 250 μm (L)×250 μm (W)×50 μM (H) with a 50 μm inter-pallet gap. The post dimensions are 60 (L)×60 (W)×100 (H) μm.

FIG. 3. Culture and replication of HeLa cells. A) Brightfield image of a small region of a pallet array with colonies of HeLa cells after 72 h in culture. B) Brightfield image of the pallet array mated with the printing array. The focal plane is at the contact plane of the posts with the pallet array. C-F) Brightfield and fluorescence images of HeLa cells stained with the viability dye calcium red-orange present on corresponding regions of the pallet array (C,D) and the printing array (E,F) after the arrays have been mated for 24 h and then separated. Cells can be seen on the pallets of both arrays as well as along the posts. G-J) Localization of eGFP-expressing and wild-type colonies on the arrays. Shown are brightfield and fluorescence images of corresponding regions of pallet and printing arrays with replicated colonies after the arrays had been mated for 24 h and then separated. In G and H, 3 colonies are seen only one of which is composed of cells expressing eGFP. In I and J, the replicated colonies are seen to be composed of the same phenotypes. Note that the cells from the colony in the lower center pallet are on the post and have not yet spread to the printing pallet.

FIG. 4. Histogram plots of the replication efficiency as various parameters are changed. A) Replication efficiency vs. post diameter for HeLa cells after the arrays had been mated for 24 h. B) Replication efficiency vs. post height for HeLa cells after the arrays had been mated for 24 h. C) Replication efficiency vs. cell type after the arrays had been mated for 24 h.

FIG. 5. Printing of IA32 cells, A-D) Brightfield and fluorescence images of corresponding regions of a pallet array (A,B) and a printing array (C,D) separated after having been mated for 24 h. The IA32 cells have been stained with the viability dye calcium red-orange.

FIG. 6. Schematic illustration of a first embodiment of an apparatus of the invention, including indexing elements formed on both the culture plate and replication plate.

FIG. 7. Schematic illustration of a second embodiment of an apparatus of the invention, including indexing elements connected to both the culture plate and replication plate. (A) The system consists of two cassettes precisely fabricated by a CNC machine. (B) The two cassettes are mated accurately guided with four V-shaped grooves. (C) The pallet array is assembled precisely to the pallet cassette guided by 1002F structures (denoted by *). (D) The printing array is assembled precisely to the printing cassette guided by 1002F structures (denoted by #).

### DETAILED DESCRIPTION OF THE PREFERRED EMBODIMENTS

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

Like numbers refer to like elements throughout. In the figures, the thickness of certain lines, layers, components, elements or features may be exaggerated for clarity. Where used, broken lines illustrate optional features or operations unless specified otherwise.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

Spatially relative terms, such as “under,” “below,” “lower,” “over,” “upper” and the like, may be used herein for ease of description to describe an element's or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus the exemplary term “under” can encompass both an orientation of over and under. The device may otherwise be oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only, unless specifically indicated otherwise.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section. Thus, a first element, component, region, layer or section discussed herein could be termed a second element, component, region, layer or section without departing from the teachings of the present invention. The sequence of operations (or steps) is not limited to the order presented in the claims or figures unless specifically indicated otherwise.

“Cells” for carrying out the present invention are, in general, live cells, and can be any type of cell, including animal (e.g., mammal, bird, reptile, amphibian), plant (including monocot and dicot), or other microbial cell (e.g., yeast, gram negative bacteria, gram positive bacteria, fungi, mold, algae, etc.).

“Liquid media” for carrying out the present invention, in which cells are carried for depositing on an array as described herein (and specifically within the cavities of the microcups) may be any suitable, typically aqueous, liquid, including saline solution, buffer solutions, Ringer's solution, growth media, and biological samples such as blood, urine, saliva, etc. (which biological samples may optionally be partially purified, and/or have other diluents, media or reagents added thereto).

“Interdigitated” as used herein with respect to carriers or microcups in an array means that the pattern of the array is staggered or off-set (typically in a uniform or repeating pattern) so that gap intersections are reduced in size and the opportunity for cells to settle at such intersections is reduced. Interdigitation can be achieved by one or more of a variety of means. The microcups can be hexagonal or triangular in cross-section; the microcups, when square or rectangular, can be offset from one another in adjacent row. The microcups can be provided with one or more vertical ridges that, when arranged in an array, interdigitates with gaps between microcups in adjacent rows. Numerous variations on the foregoing will be apparent to those skilled in the art.

“Flexible substrate” as used herein is, in general, a flexible or elastomeric substrate, and may be conveniently formed from a material in which cavities may be produced and the carrier molded directly therein. Examples include, but are not limited to, silicones (e.g., polydimethylsiloxane (or “PDMS”), Silastic, Texin and ChronoFlex silicone materials), polyurethane substrates, styrene-butadiene copolymer, polyolefin and polydiene elastomers, thermoplastic elastomers, other biomedical grade elastomers, etc.

“Biodegradable polymer” as used herein includes biodegradable polyesters and biodegradable aliphatic polymers. Numerous examples are known, including but not limited to those described in U.S. Pat. Nos. 7,879,356; 7,862,585; 7,846,987; 7,842,737; and 7,767,221. Particular examples include, but are not limited to, polymers that includes poly(lactic acid) (including poly(L-lactide) and poly(DL-lactide)), polyglycolide, poly(lactide-co-glycolide) (PLGA) (including poly(DL-lactide-co-glycolide)), poly(caprolactone) (PCL), poly[(R)-3-hydroxybutyric acid (PLA), poly(glycolic acid) (PGA), poly(ethylene glycol) (PEG), poly(hydroxy alkanoates) (PHA), dendritic polymers with acidic, hydroxyl and ester functional groups, modified polyesters, acetylated cellulose, starch, a starch derivative, a co-polymer of PLA and a modified polyester, or a combination thereof.

“Hydrogel” as used herein refers to a composition comprising a network of natural or synthetic polymer chains that are hydrophilic, and in which a significant amount of water is absorbed. Numerous examples are known, including but not limited to those described in U.S. Pat. Nos. 7,883,648; 7,858,375; 7,858,000; 7,842,498; 7,838,699; 7,780,897; and 7,776,240.

### Cell Culture Plates or Arrays.

Cell culture plates used to carry out the present invention typically comprise a first substrate and a plurality of cell carriers on said substrate in a first pattern. Cell culture plates are known and the particular culture plates, including culture plates formed on rigid substrates, useful for carrying out the present invention can be produced in accordance with known techniques with modifications as necessary for the present invention as will be apparent to those skilled in the art. (see, e.g., Allbritton et al., U.S. Pat. No. 7,759,119; Bachman et al., U.S. Pat. No. 7,695,954; Wang et al., US Patent Application Pub. No. US2007/0128716; Allbritton et al., WO 201/093766; Allbritton et al., WO 2011/103143; and Allbritton et al., WO2010/068743).

Indexing elements are preferably included on the cell culture plates in some embodiments of the invention, as discussed further below.

Flexible Substrate Arrays.

As noted above, in some embodiments of the present invention the culture plate is generally comprised of a common substrate formed from a flexible resilient polymeric material and having a plurality of wells formed therein; and a plurality of rigid cell carriers releasably connected to the common substrate, with said carriers arranged in the form of an array, and with each of the carriers resiliently received in one of said wells.

The cavities in said substrate can be separated by walls. The walls may be uniform or non-uniform and of any suitable dimension. In some embodiments, the walls have an average width of at least 2 micrometers, up to 5, 10, 100, 200, 500, or 1000 micrometers. In general, the walls have an average height of at least 2 or 5 micrometers, up to 200, 500, or 1000 micrometers.

The cavities in the substrate in some embodiments have floors. The floors can be uniform or non-uniform and of any suitable thickness. In some embodiments, the floors have an average thickness of from 2 or 5 to 200 or 500 micrometers.

In other embodiments, the floor is eliminated and the cavity is a continuous opening from the top surface of the substrate to the bottom surface of the substrate. Such arrays can be made in accordance with known techniques by, for example, from the substrate with such continuous cavities on top of a release layer.

The array may be in any suitable uniform or non-uniform arrangement, including but not limited to interdigitated arrays and/or or tilings.

The substrate has a top surface, and the carriers are preferably positioned either below the top surface, or up at (that is, even with, or flush with) the top surface). Preferably the carriers do not protrude above the top surface of the substrate. This configuration can follow from one preferred way of making the array, by forming the substrate with the cavities and then casting the carriers in the cavities, as discussed further below.

The carriers are configured to release from said substrate upon mechanical distortion of said substrate: that is, the application of a gradual energy such as mechanical pushing or continuous vibration, in contrast to a “burst” of energy, as discussed further below. The carriers or rafts may be in any suitable geometry, including cylindrical, elliptical, triangular, rectangular, square, hexagonal, pentagonal, octagonal, etc., including combinations thereof. In some embodiments, the carriers have heights of at least 2 micrometers, up to 400 or 500 micrometers. In some embodiments, the carriers have maximum widths of at least 5 or 10 micrometers, up to 1000 micrometers.

The substrate can be produced by any suitable technique, such as printing or microprinting. The carriers can likewise be produced by any suitable technique, such as by casting the carriers in the cavities or wells formed during printing of the substrate. In some embodiments, the carriers have a concave top surface portion. While any desired physical or structural feature can be incorporated into the carrier top portion, alone or in combination, a concave top surface portion is conveniently formed by meniscus coating of the side walls of said wells or cavities in the substrate during the process of casting said carriers in those cavities or wells.

The carriers (also referred to as “rafts” herein) can be formed of any suitable material. The rafts are, in some embodiments, preferably transparent or semitransparent (e.g., visually transparent, optically transparent, optically transparent at certain wavelengths, and/or optionally containing elements or features that magnifies, reflects, refracts, absorbs or otherwise distorts light or certain wavelengths of light as light passes therethrough, etc.) A variety of polymers and other materials can generally satisfy the requirements for the microcarriers or rafts. Currently polystyrene (including copolymers thereof) and epoxy are preferred. A wide range of epoxies can be used including the epoxy novolac resins such as EPON 1001F, 1009F, and 1007F. These resins can be used alone or with crosslinkers. Preformulated epoxies, such as Loctite Hysol and other medical device epoxies can also be used. Medical device polymers such as polystyrene (including copolymers thereof, such as poly(styrene-co-acrylic acid) (PS-AA)), poly(methyl methacrylate), polycarbonate, and cyclic olefin copolymer can also be used as raft materials. Sol-gel materials, ceramics, and glasses (e.g., sodium silicate) can also be used as raft materials. Biodegradable polymers and hydrogels can also be used as raft materials. The rafts may be formed of a single material, may be a composite of two or more layers of different materials, etc. The rafts may be “doped” with one or more additional agents, such as growth factors (e.g., as in matrigel), magnetic or ferromagnetic particles or nanoparticles, live feeder cells, etc.

### Cell Replication Plates or Arrays.

Cell replicating (or “printing” plates) generally comprise a second substrate and a plurality of cell sampling posts on said second substrate in a second pattern that preferably corresponds to the first pattern so that, when the cell culture plate and cell replication plate are brought into oppositely facing alignment (e.g., substantially parallel with one another), and with each of the sampling posts align with a respective one of the cell carriers.

The second substrate may be a rigid or flexible substrate as described above in connection with the cell culture plates.

Posts may be formed from the same material as either the substrates or cell carriers described in connection with cell culture plates above.

Posts may be formed in any suitable shape and configuration. Posts may be tapered or of uniform cross-section throughout their height. Posts may have any suitable cross-section, including circular, elliptical, triangular, square, pentagonal, hexagonal, star-shaped, etc. Channels may be formed in the side walls of the posts to facilitate cell growth therein.

The posts are aligned with the carriers in a position in which cells growing on the carrier can proliferate onto the post. The posts may directly contact the carrier, or may be spaced apart slightly from the carrier (so long as the space is sufficiently small to permit a cell colony growing on the carrier to proliferate onto the post). Posts may configured so that they are centrally aligned with corresponding carriers, offset from the center axis of corresponding carriers, or even positioned adjacent corresponding carriers.

Indexing elements are preferably included in some embodiments of the replication plates of the present invention, as discussed further below.

### Indexing Elements.

The cell culture plates and the replication plates preferably each have an indexing element formed thereon or connected thereto. The indexing element of the cell culture plate and the indexing element of the replication plate is configured to correspond with one another and cause the posts to be held in proper orientation and configuration with the corresponding cell carrier so that a “reverse template” of the plurality of cell colonies on the first substrate is formed on the second substrate.

Any suitable corresponding pair of indexing elements may be used, so long as they are configured to align the posts with the carriers when the cell culture plate and the replication plate are brought into oppositely facing alignment, including nesting ring portions or edge portions; one or more corresponding tongue and groove elements (of any suitable cross-sectional shape, including V-shaped, half circle, square, etc). In some embodiments, each pair of indexing elements includes a first pair of oppositely facing opposing surface portions to prevent rotation of the assembled carriers in the clockwise direction, and a second pair of oppositely facing opposing surface portions to prevent rotation of the assembled carriers in the counterclockwise direction. The first and second pairs of opposing surface portions may be continuous (e.g., the opposite edge portions of an elliptical surface), discontinuous (e.g., the opposite edge portions of a V-shaped tounge-and-groove system). The opposing surfaces may be vertical or offset from vertical, so long as they are at sufficient angle from the faces of the two carriers as substantially inhibit, constrain, or prevent rotation thereof, when the carrier array and replication arrays are assembled). In some embodiments, the at least one pair of indexing elements are symmetric, so that the cell culture plate and the replication plate can be brought into oppositely facing alignment in more than one position; in other embodiments; the at least one pair of indexing elements are asymmetric, so that the cell culture plate and the replication plate can be brought into oppositely facing alignment in only one position (e.g., so that correspondence between cells on posts and cells on carriers can be conveniently ascertained after the replication plate and the culture plate are separated)

### Methods of Use.

The present invention provides a method for selecting and propagating a cell colony of interest from among a plurality of primary cell colonies carried on a first substrate in culture, the method comprising the steps of: (a) generating a plurality secondary cell colonies corresponding to the of the plurality of primary cell colonies as a reverse template thereof on a second substrate; (b) selecting at least one secondary cell colony of interest from among the plurality of secondary cell colonies on the second template; (c) analyzing the at least one cell colony of interest to confirm the presence or absence of a desired feature therein; (d) identifying at least one primary cell colony corresponding to the at least one secondary cell colony of interest; then (d) propagating the at least one primary cell colony when the desired feature is present in the corresponding at least one secondary cell colony.

In some embodiments, the generating step is carried out in culture (e.g. in a liquid growth or culture medium that maintains and/or promotes the growth of the cells).

In some embodiments, the generating step is carried out by: providing discrete pallets on the first substrate on which the primary cell colonies grow; providing discrete posts on the second substrate, and positioning the posts sufficiently adjacent the pallets so that primary colonies growing on the discrete pallets form corresponding secondary colonies on the discrete posts.

In some embodiments, the generating step is carried out by positioning the first substrate and the second substrate in an oppositely facing configuration.

In some embodiments, the at least one secondary cell colony of interest comprises a plurality (e.g., some, or all) of the secondary cell colonies.

In some embodiments, the analyzing step is carried out by destructive analysis, examples of which include, but are not limited to, procedures which include or comprise: polymerase chain reaction amplification, intracellular immunostaining, mass spectrometry, mRNA expression, electron microscopy, electrophoretic analysis, and DNA analysis.

In some embodiments, the destructive analysis is carried out on a plurality of selected secondary cell colonies simultaneously.

In some embodiments, the desired feature is stable expression of a heterologous nucleic acid.

In some embodiments, the plurality of cell colonies comprises not more than 10 colonies; in other embodiments the plurality of cell colonies comprises at least 50 colonies.

In some embodiments, the cell colony of interest comprises not more than 10, 100, 1000, or 10,000 cells.

The present invention is explained in greater detail in the following non-limiting Examples.

### Examples

The current work describes a new technique to both achieve efficient and simultaneous splitting of multiple colonies in a single step combined with parallel destructive assay of a molecular characteristic to identify desired colonies. In one embodiment, the platform mates two matching arrays of three-dimensional microstructures. One array is composed of a high density array of pallets and is referred to as the pallet array. A second array is composed of similar microstructures in an identical pattern to the pallet array, but which also possesses posts at center of each pallet which serve to connect each pallet of the two arrays. The posts enable cell migration from colonies on the pallet array to the printing array. After colonies are established on the pallet array, the arrays are mated to allow cell migration. Separation of the two arrays split the colonies to create a patterned replica of the original colonies. The printing array was then subjected to a destructive assay, which identified the molecular characteristic defining target colonies. The corresponding parent colony was then isolated for further use.

### Experimental Section

Materials.

EPON resin 1002F phenol, 4,4′-(1-methylethylidene)bis-, polymer with 2,2′-[(1-methylethylidene)bis(4,1-phenyleneoxymethylene)]bis(oxirane) was obtained from Miller-Stephenson (Sylmar, Calif.). UVI-6976 photoinitiator (triarylsulfonium hexafluoroantimonate salts in propylene carbonate) was purchased from Dow Chemical (Torrance, Calif.). SU-8 developer (1-methoxy-2-propyl acetate) was purchased from MicroChem. Corp. (Newton, Mass., USA). All other photoinitiators and resins were from Sigma-Aldrich (St. Louis, Mo.) as was γ-butyrolactone (GBL). (Heptadecafluoro-1,1,2,2-tetrahydrodecyl) trichlorosilane was from Gelest Inc. (Morrisville, Pa.). Poly(dimethylsiloxane) (PDMS) (Sylgard 184 silicone elastomer kit) was purchased from Dow Corning (Midland, Mich.). Dulbecco's Modified Eagle Medium (DMEM), fetal bovine serum (FBS), 0.05% Trypsin-EDTA and penicillin/streptomycin were obtained from Invitrogen (Carlsbad, Calif.). Fibronectin, glass microscope slides and all other reagents were obtained from Fisher Scientific (Pittsburgh, Pa.). The microfabrication masks were drawn using TurboCAD (IMSI/Design, LLC, Novato, Calif.) and then printed and fabricated by Fineline Imaging (Colorado Springs, Colo.).

Fabrication of the Array.

Fabrication of the pallet and the printing arrays used a standard process previously described. Briefly, a layer of 1002F-50 (120 μm thick for the pallet array and 50 μm for the printing array) was spin-coated on a 75 mm×50 mm glass microscope slide, and then baked for 20 min on a hotplate at 65° C. followed by a second bake (90 min for pallet and 60 min for the printing array) at 95° C. After soft baking, a chrome mask was placed on top of the 1002F-50 film on the slide and exposed using a Newport 97485 UV exposure system. The slide was then baked for 1 min at 65° C. and 8 min at 95° C. for hard baking. The slide was allowed to cool in air to room temperature. After cooling, a layer of 1002F-50 which varied depending on the desired height of the structures for the particular experiment (i.e. 50 μm for the pallet array and 20-120 μm for the printing array) was spin-coated on the slide and the slide was baked for 20 min on a hotplate at 65° C. followed by another bake at 95° C. (60 min for the pallet and 90 min for the printing array). After this second soft bake, the second chrome mask was placed on top of the slide, aligned and exposed using an aligner system (MA6, SUSS Microtec, Germany). The slide was then baked for 1 min at 65° C. and 8 min at 95° C. for hard baking. Finally, the slide was immersed in SU-8 developer for 10 min, sprayed with isopropyl alcohol (IPA) and dried with compressed nitrogen. The array was baked at 95° C. for 5 min and then baked at 120° C. for 1 hr to completely remove any remaining solvent from the photoresist.

The dimensions of the individual pallet elements forming the array were 150 μm (L)×150 μm (W)×120 μm (H) with a 150 μm gap between the pallets. The array contained 3000 pallets. Two cross structures each with dimensions of 8 mm (L)×5 mm (W)×170 μm were created at the edges of the array for alignment purposes. For the printing array, the dimensions of the base pallet were 250 μm (L)×250 μm (W)×50 μm (H) with a 50 μm inter-pallet gap. The dimensions of the printing post varied depending on the experiment (see Results). The optimal post size was 30 μm (L)×30 μm (W)×100 μm (H) with a 270 μm inter-post gap. The printing array also contained 3000 pallets/posts aligned to match those on the pallet array. Two alignment structures that mated with those on the pallet array were created at the edges of the array whose outside dimensions were 1.2 cm (L)×8 mm (W) with a height that varied depending on the height of the posts on this array (see Results). This structure contained an inner groove with dimensions of 8.06 mm (L)×5.06 mm (W)×50 μm (deep).

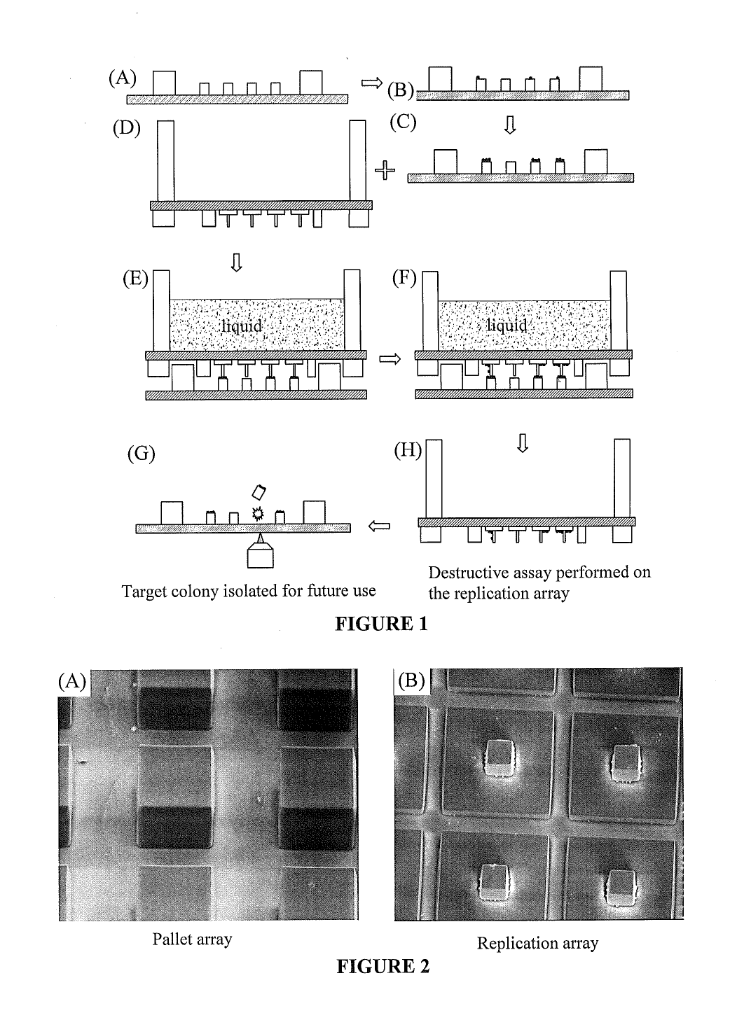

After microfabrication, both the pallet and printing arrays were treated with perfluoroalkyl silane to generate hydrophobic regions between the pallet structures that enabled the creation of a “virtual wall” formed by a continuous air bubble entrapped between the pallets when the array was immersed in media. This region of air is needed to restrict cells to the pallet top surface as previously described.{Wang, 2007 #678} Before use, a PDMS ring surrounding the array was constructed to provide a chamber for housing the cells and media. The PDMS ring was formed by putting a square plastic mold sized to match the array dimensions inside a 35-mm diameter polystyrene tissue culture dish. The Sylgard 184 resin with curing reagent (ratio 10:1) was then poured into the dish and the assembly was heated in an 80° C. oven for 20 min. The solid mold was extracted after cooling. For the pallet array, the PDMS ring was removed and attached to the array by simply pressing the PDMS against the glass. For the printing array, the PDMS ring was glued to the back side of the array (see FIG. 1). Before use, the array was sterilized by soaking in 70% ethanol for 20 min and then blown dry with compressed nitrogen.

Fabrication of the collection plate. To fabricate a device for collection of released pallets with colonies, an array (80 columns; 80 rows) of 250 μm (L)×250 μm (W)×70 μm (H) structures was fabricated on a 2×3 inch glass substrate which was then silanized as described above. After silanization, this array was used as the mold for fabricating an array of PDMS wells for collection. A thin layer (about 2 mm) of uncured PDMS was poured onto the mold and heated in an oven at 80° C. for 20 min, after which the assembly was taken out and the PDMS film was peeled off the mold. A PDMS ring (2.0×2.0 cm) was glued to the film to form a reservoir as described above. Before use, the collection well plate was sterilized in 70% ethanol for 20 mM followed by wash in phosphate buffered saline (PBS: 135 mM NaCl, 3.2 mM KHPO₄, 0.5 mM KH₂PO₄ and 1.3 mM KCl; pH=7.4)×5. Culture medium was added just before use.

Laser-based release of microstructures from the array. A pulsed laser was used to release the pallets from the array as has been described in detail previously.{Salazar, 2008 #31} Briefly, a laser pulse (5 ns, 532 nm) from a Q-switched Nd:YAG laser (Minilite I, Continuum Electro-Optics Inc., Santa Clara, Calif.) was focused by a 40× microscope objective at the interface of the glass substrate and one of the pallets (see Results). The focused pulse led to formation of a plasma and cavitation bubble. The expansion of the cavitation bubble at the base of the pallet mechanically dislodged it in an upward direction.{Quinto-Su, 2008 #51}

Cell Plating and Culture on the Pallet Array.

HeLa, a human cervical carcinoma cell line; NIH 3T3, a murine fibroblast cell line; IA32, a mouse embryonic fibroblast cell line; A549, a human alveolar adenocarcinoma cell line; and HT1080, a human fibrosarcoma cell line, were used in the current studies. Both wild type and a molecularly engineered HeLa cell line stably expressing a nuclear green fluorescent protein (GFP) fused with the histone H1 protein were utilized. Cells cultured on the array and those on pallets released from the array were cultured in conditioned media.{Wang, 2007 #679} The base medium used was DMEM with 10% FBS, L-glutamine (584 mg/L), penicillin (100 units/mL) and streptomycin (100 μg/L). The arrays were sterilized by immersion in ethanol for 20 min and then allowed to dry in a cell culture hood prior to use. To enhance cell attachment and growth, the arrays were coated before use with fibronectin (5 μg/mL for pallet and 15 μg/mL for the printing array) in PBS by incubation in the fibronectin solution for 30 min at room temperature. The arrays were washed with sterile deionized water ×4 with a final rinse in media before use. To plate cells on the array, 2500 cells suspended in 1 mL of media were added to the chamber surrounding the array and allowed to settle. The cell number was chosen empirically to provide ≦1 cell per pallet in order to create clonal colonies. Plated cells were cultured in a humidified, 5% CO₂ atmosphere at 37° C.

Cell Replication.

After cells were plated on the pallet array, they were cultured for 72 hr to allow small clonal colonies to form. The PDMS ring was removed under sterile conditions and media was added to the Petri dish containing the array such that the level of the media was mm above the array. The fibronectin pre-coated printing array was then placed in contact with the pallet array with the patterned side facing the pallet array. Using manual placement with the aid of alignment structures, the posts of the printing array were positioned near the center and in contact with the pallets on the pallet array. Four milliliters of sterile fluid was then added to the chamber formed by the PDMS ring on the backside of the printing array to weight the array in order to keep it in position. The mated arrays were returned to a standard tissue culture incubator. After 24 hr, the two arrays were separated by hand under sterile conditions. Immediately upon separation, the printing array was immersed in media (10 mL media in a 100 mm Petri dish). Both the pallet and the printing arrays were maintained in media and then imaged to identify the percentage of replicated colonies or to carry out viability assays. Unless otherwise specified, in each experiment 3 identical arrays and 50 positions per array were analyzed to generate the data.

Cell Collection and Culture after Release.

The pallet array was rinsed with fresh, pre-warmed culture media (37° C.) ×3 before the release procedure. After laser-based release, individual pallets with cells were collected as previously reported.{Wang, 2007 #679} The collected cells were maintained in freshly prepared conditioned media for expansion. To identify and track the released pallets, the array was fabricated with numbered pallets as previously described.{Wang, 2007 #679}

### Results and Discussion

Array Design and Fabrication.

The design employed the mating of two matching arrays of three-dimensional microstructures (FIG. 1). A pallet array was composed of square posts created from a biocompatible photoresist 1002F (FIG. 1, 2A).{Pai, 2007 #53;Wang, 2007 #678} A second array or “printing array” consisted of an array of larger square pallets aligned in register to those on the pallet array. The printing array pallets each possessed a square 1002F post fabricated at their center (FIG. 1, 2B). These posts served as a bridge between the elements of the two arrays when the pallet and printing arrays were mated. A two-step photolithography process was used to create both arrays. For the pallet array, the two-step process was required because the alignment structures (see below) were taller than the pallets. The printing array was fabricated in a two-step process to create the base pallets with the attached posts and the corresponding mating structure used in alignment. Both arrays contained 3000 elements positioned so that the centers of the pallets on each array were axially aligned when the arrays were mated (FIG. 1E). The individual pallets of the pallet array were of smaller diameter (150 μm) than those forming the printing array (250 μm). The dimensions of the printing post atop each pallet of the printing array varied depending on the experiment (see below). In all experiments, the overall dimensions of both arrays were ˜1.5×1.5 cm. The lanes between the pallets on both arrays were coated with a perfluoroalkyl silane. The hydrophobic cavity between the pallets entrapped air creating a “virtual wall” between the pallets which acts to localize cells to their initial pallets.{Wang, 2007 #678}

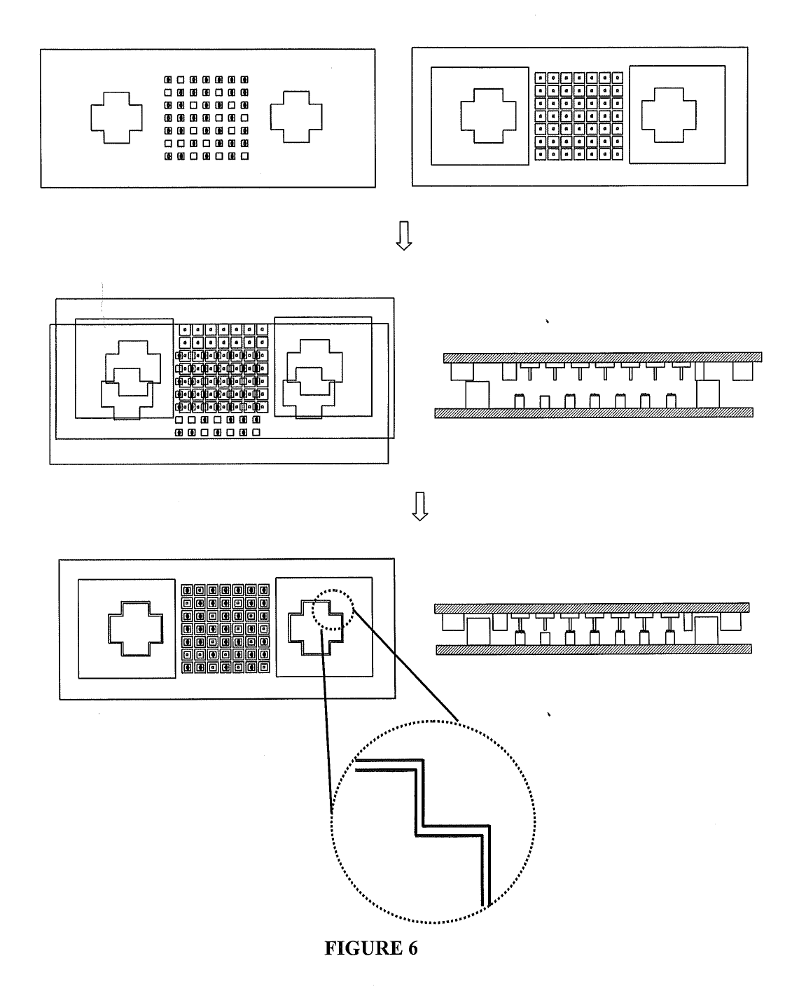

An important aspect of this procedure was the design of an easy and accurate manual alignment system. This was accomplished by incorporating larger cross-shaped structures at the edge of the arrays that fit together in a “tongue and groove” manner to provide precise and simple manual alignment when mating the two arrays (FIG. 6). These structures fit together as the two arrays were positioned by hand to place each printing post near the center of each pallet. The tongue side of the alignment structures was created on the pallet array and was 8 mm (L)×5 mm (W).

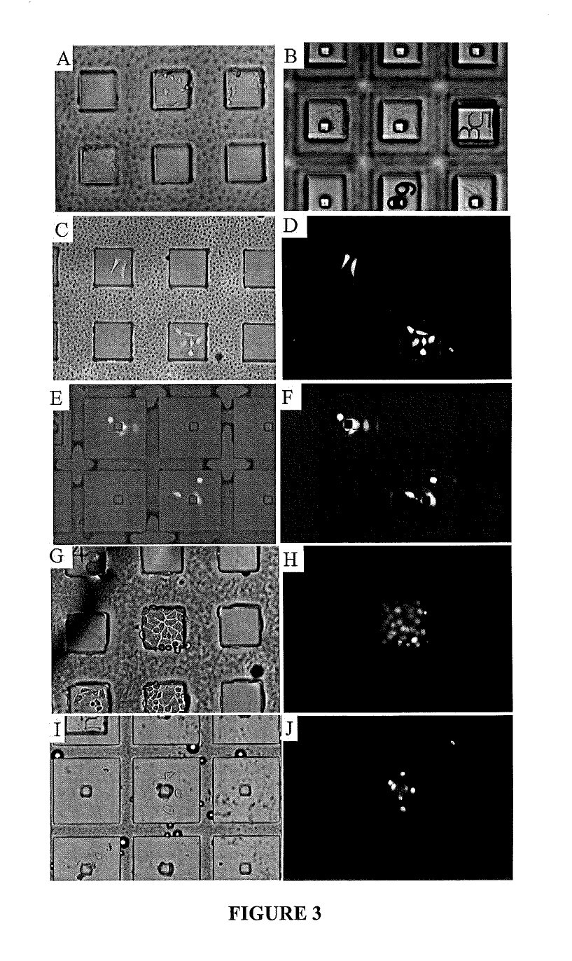

The height of this structure was fixed at 170 μm. The grooved structure was placed on the printing array. The outside dimensions of this structure were 1.2 cm (L)×8 mm (W). The height varied from 70 to 170 μm depending on post height in order to allow the printing posts to come into contact with the pallets when the arrays were mated. The inside dimensions of the groove were 8.06 mm (L)×5.06 mm (W)×70−170 μm (deep). When the two alignment structures were mated, the tolerance for post positioning was 30 μm from the pallet center in any one direction (FIG. 3B and FIG. 6).

Colony Printing.

To determine whether cells growing on the pallets could migrate across the posts to the printing array, HeLa cells (2500 cells) were plated then cultured on a pallet array for 72 h to allow colonies to develop (FIG. 3A). At that time the pallets contained on average 9±3 cells per colony. A printing array possessing 30 μm posts was then mated to the pallet array (FIG. 3B) and the paired arrays were returned to culture. After 24 h, the paired arrays were separated and examined microscopically (FIG. 3C,D). When care was taken in mating the arrays to eliminate sliding of the surfaces across one another, cells were present on the printing array sites only when a corresponding colony was present on the pallet array. Cells were observed to be present along the posts and on the base pallets of the printing array in registration with the colonies of the pallet array (FIG. 3E,F). Of the pallet/post sites on the replication array, 35±16% contained cells on the posts alone, 12%±4% solely on the pallet, and 53%±19% possessed cells on both post and pallet. These data support that sites were not seeded from cells that were shed from random portions of the array. The cells on both arrays were viable (100±0%) as determined by staining with the viability dye calcium red-orange.

To further evaluate the accuracy of colony replication, fluorescent HeLa cells stably transfected with eGFP were mixed with wild type HeLa cells at a ratio of 1:10 and plated on a pallet array at <1 cell/pallet. After 72 hr in culture, the pallet and printing arrays were mated and returned to culture. After 24 h, the two arrays were separated and maintained in culture for an addition 24 h. The separated pallet and printing arrays were then imaged under fluorescence and brightfield microscopy (FIG. 3G-J). The printing arrays were screened for sites in which fluorescent cells were present on one post/pallet with non-fluorescent cells present on one or more adjacent posts/pallets. These sites were compared with the corresponding sites on the pallet array. In every instance, the fluorescent signatures of the cells at each site on the printing array were identical to that of the corresponding colonies on the pallet array (n=3 arrays, 8 sites analyzed/array). Importantly, no colonies on either array were noted to be a mixture of fluorescent and non-fluorescent cells. These data indicated that the clonal colonies that were formed on the printing array faithfully represented their corresponding colonies on the pallet array. These findings also suggest that cells transferred to the printing array only by migration from the pallet array across the printing post.

Impact of Post Dimensions and Cell Type on Replication Efficiency.

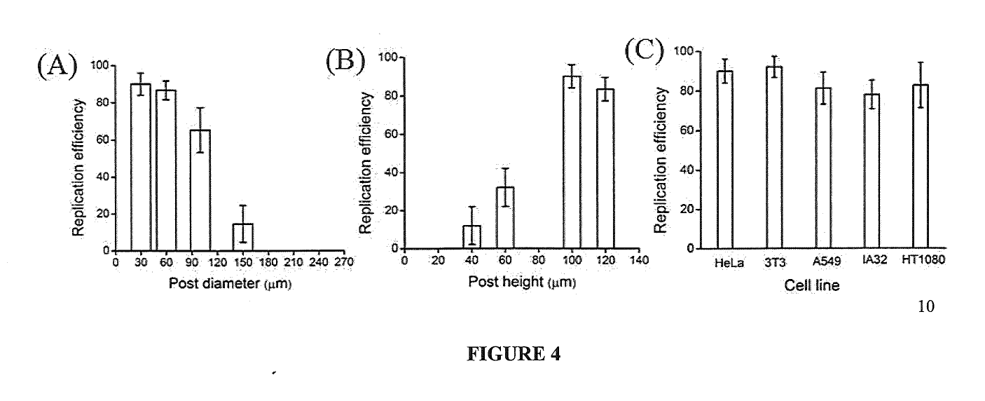

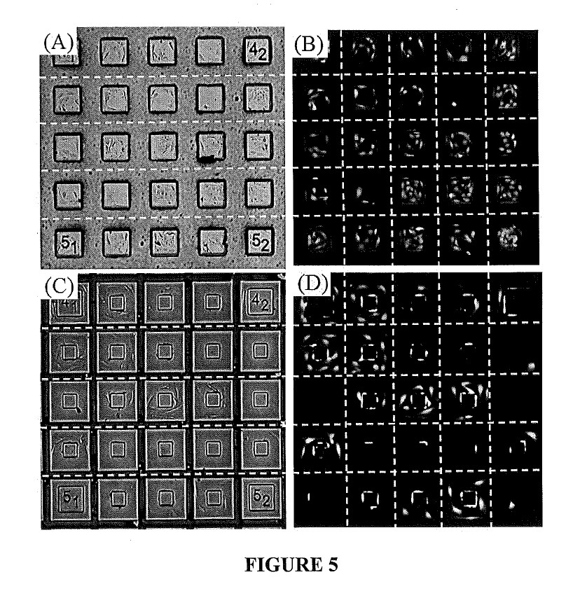

A series of experiments were conducted to assess whether the dimensions of the intervening post affected migration of cells from the pallet array to the printing array. In each of these experiments, HeLa cells were plated on the pallet array at ≦1 cell/pallet and then cultured for 72 h at which time the printing array was mated as described above. After 24 h, the arrays were separated and analyzed for colony extension onto the printing array. In the initial series of experiments, printing arrays possessed 100-μm tall posts with the post diameter varying between 30-260 μm. Only when the diameter of the post was less than the diameter of the pallet on the pallet array were cells present on corresponding posts and pallets on the printing array (FIG. 4A). Smaller post diameters were more efficient at enabling transfer of cells, as demonstrated with the 30 μm post arrays which had on average 90%±6% of mated pallets showing transfer of cells from the pallet array to the printing array.

A second series of experiments was then performed in which the post diameter was constant (30 μm), while post height was varied between 20-120 μm (FIG. 4B). Somewhat surprisingly, in these experiments the efficiency of cell transfer between the two arrays was greatest at the longer post lengths tested with a transfer rate of 0%±0% for the arrays with 20 μm posts and 91%±6% with 100 μm posts. This finding is likely the result of diminished diffusion of nutrients and oxygen that impair cell growth when the distance between the two arrays is reduced below 100 μm.

Using the optimized post dimensions of a 30-μm diameter and 100-μm length, the efficiency of colony replication was evaluated with five different cell lines (HeLa, 3T3, A549, HT1080, and IA32). In four independent experiments, each cell type was plated on a pallet array at 1 cell/pallet and cultured for 72 h. The printing array was mated for 24 h and then the arrays were separated and analyzed. While there was some variability depending on cell type, all five cell types were efficiently transferred with replication rates between 78-92% (FIG. 4C).

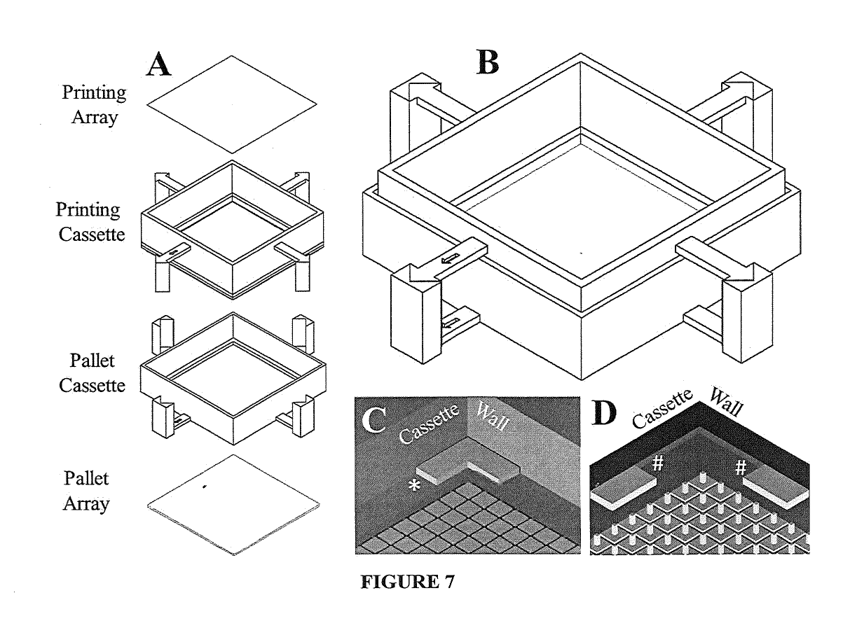

An additional embodiment of the present invention is shown in FIG. 7. Cells are cultured on the pallet array in the cassette. At the time for array mating, the two cassettes are mated by the guidance of four symmetric indexing elements having V-shaped grooves formed therein (FIG. 7B), which bring the replication array into contact with the culture array. The culture array has corresponding indexing elements having V-shaped guides formed thereon that match the V-shaped grooves (since the array is symmetrical the indexing elements are identical permitting the arrays to be brought together into alignment in any of four positions; if the arrays are non-symmetrical, then an irregularity can be included in the indexing elements (for example, one pair of groove and guide replaced with elements of a different cross-sectional shape, such as a square, half circle, 1-beam, etc.)). Medium is added onto the replication cassette for either perfusion and/or to apply pressure. The mating and separation of cassettes is performed in a tissue-culture hood. The mated cassettes are placed in a 100×25 mm deep petri dish for sterility. The x-y alignment accuracy between the cassettes is controlled to approximately within ±10 μm since the dimensional accuracy of each cassette is within approximately ±5 μm. The x-y alignment accuracy between two arrays is approximately ±20 μM: ±5 μm from pallet array/pallet cassette, approximately ±5 μm from printing array/printing cassette, and approximately ±10 μm from pallet cassette/printing cassette.

### CONCLUSIONS

This platform for sampling and identifying cell colonies is expected to considerably reduce the time, manpower and reagent costs imposed by conventional approaches for clonal selection by colony picking and destructive assay.

The foregoing is illustrative of the present invention, and is not to be construed as limiting thereof. The invention is defined by the following claims, with equivalents of the claims to be included therein.

That which is claimed is:

1. An apparatus comprising, in combination:
(a) a cell culture plate, said cell culture plate comprising a first substrate and a plurality of cell carriers on said substrate in a first pattern;
(b) a cell replication plate, said cell replication plate comprising a second substrate and a plurality of cell sampling posts on said second substrate in a second pattern corresponding to said first pattern, each of said sampling posts configured to align with a respective one of said cell carriers in a position in which cells growing on said cell carrier propagate onto said sampling post;
so that a plurality of distinct cell colonies growing on said cell culture plate are replicated on said cell replication plate.

2. The apparatus of claim 1, wherein said cell culture plate and said cell replication plate are positioned on one another, with said cell carriers and said sampling posts facing one another.

3. The apparatus of claim 2, wherein said cell culture plate and said cell replication plate each have an indexing element formed thereon or connected thereto for aligning said cell carriers and said sampling posts.

4. The apparatus of claim 1, wherein said cell carriers are releasably connected to said substrate.

5. The apparatus of claim 1, wherein said first substrate is flexible.

6. The apparatus of claim 1, wherein said cell carriers are rigid.

7. The apparatus of claim 1, wherein said cell carriers are transparent.

8. The apparatus of claim 1, wherein said cell carriers comprise microcups.

9. The apparatus of claim 1, wherein said cell culture plate further comprises:
trapped gas regions on said substrate, with said plurality of cell carriers separated from one another by said trapped gas regions; and/or
walls on said substrate, with said plurality of cell carriers separated from one another by said walls.

10. The apparatus of claim 1, wherein:
said cell carriers have heights of at least 2 micrometers, up to 500 micrometers; and/or wherein said cell carriers have maximum widths of at least 5 micrometers, up to 1000 micrometers;
said cell carriers are included on said first substrate at a density of from 0.1 to 20 carriers per square millimeter;
said posts have heights of at least 10 micrometers, up to 1000 micrometers;
and/or said posts have maximum widths at the tip thereof of at least 1 micrometer, up to 500 micrometers; and/or
said posts are included on said second substrate at a density the same as the density of said cell carriers on said second substrate.

11. A method for selecting and propagating a cell colony of interest from among a plurality of primary cell colonies carried on a first substrate in culture, said method comprising the steps of:
(a) generating a plurality secondary cell colonies corresponding to said of said plurality of primary cell colonies as a reverse template thereof on a second substrate;
(b) selecting at least one secondary cell colony of interest from among said plurality of secondary cell colonies on said second template;
(c) analyzing said at least one cell colony of interest to confirm the presence or absence of a desired feature therein;
(d) identifying at least one primary cell colony corresponding to said at least one secondary cell colony of interest; then
(d) propagating said at least one primary cell colony when said desired feature is present in said corresponding at least one secondary cell colony.

12. The method of claim 11, wherein said generating step is carried out in culture.

13. The method of claim 11, wherein said generating step is carried out by;
providing discrete pallets on said first substrate on which said primary cell colonies grow;
providing discrete posts on said second substrate, and
positioning said posts sufficiently adjacent said pallets so that primary colonies growing on said discrete pallets form corresponding secondary colonies on said discrete posts.

14. The method of claim 11, wherein said generating step is carried out by positioning said first substrate and said second substrate in an oppositely facing configuration.

15. The method of claim 11, wherein said at least one secondary cell colony of interest comprises a plurality of said secondary cell colonies.

16. The method of claim 11, wherein said analyzing step is carried out by destructive analysis.

17. The method of claim 16, wherein said destructive analysis is selected from the group consisting of polymerase chain reaction amplification, intracellular immunostaining, mass spectrometry, mRNA expression, electron microscopy, electrophoretic analysis, and DNA analysis.

18. The method of claim 16, wherein said destructive analysis is carried out on a plurality of selected secondary cell colonies simultaneously.

19. The method of claim 11, wherein said desired feature is stable expression of a heterologous nucleic acid.

20. The method of claim 11, wherein said plurality of cell colonies comprises not more than 10 colonies.

21. The method of claim 11, wherein said plurality of cell colonies comprises at least 50 colonies.

22. The method of claim 11, wherein said cell colony of interest comprises not more than 10,000 cells.

23. The method of claim 11, wherein said cell colony of interest comprises mammalian cells.

24. The method of claim 11, wherein said cell colony of interest comprises monocot or dicot plant cells.
